(19) United States
(12) Patent Application Publication
(10) Pub. No.: US 2006/0136007 A1
(43) Pub. Date: Jun. 22, 2006

(54) Deep brain stimulation apparatus, and associated methods

(76) Inventors: Marlin H. Mickle, Pittsburgh, PA (US); Steven A. Hackworth, Pittsburgh, PA (US)

Correspondence Address: ECKERT SEAMANS CHERIN & MELLOTT, 600 GRANT STREET, 44TH FLOOR, PITTSBURGH, PA, 15219, US

(21) Appl. No.: 11/312,183

(22) Filed: Dec. 20, 2005

Related U.S. Application Data
(60) Provisional application No. 60/638,037, filed on Dec. 21, 2004.

(51) Int. Cl. A61N 1/18

(52) U.S. Cl. 607/45

(57) ABSTRACT

Various methods and apparatus for providing deep brain stimulation for the treatment of diseases such as Parkinson's Disease that do not require an onboard power supply that is implanted in the patient's body. Power may be supplied from outside of the body by, for example, near-field inductive coupling with an external power supply provided in, for example, a headgear worn by the patient. Power may also be supplied by providing an antenna for harvesting ambient energy, such as ambient RF energy, and converting it into DC power. In addition, the methods and apparatus provide for remote, wireless programming of the parameters that specify the nature of electrical pulses provided to the brain via probes implanted in the brain.

### CROSS-REFERENCE TO RELATED APPLICATION

This application claims the benefit of U.S. Provisional Application No. 60/638,037, entitled “Deep Brain Stimulation,” which was filed on Dec. 21, 2004, the disclosure of which is incorporated herein by reference.

### GOVERNMENT CONTRACT

This work was supported in part by a grant from the National Science Foundation under Contract No. EEC 0203341. The United States government may have certain rights in the invention described herein.

### FIELD OF THE INVENTION

The present invention relates to methods and apparatus for providing treatment for the symptoms of various diseases, such as Parkinson's Disease (tremors), and in particular to improved methods and apparatus for providing deep brain electrical stimulation.

### BACKGROUND OF THE INVENTION

Parkinson's Disease is a neurodegenerative disorder that causes muscular tremors, stiffness, and slowness of movement. The first line of treatment for Parkinson's is the administration of drugs. Over a period of time, these drugs slowly lose their effect to arrest the symptoms associated with Parkinson's. Once a patient enters a refractory stage of the disease in which drugs are not effective, one alternative treatment option to reduce associated tremors is Deep Brain Stimulation (DBS). DBS can also be used as a part of a treatment plan for other diseases, such as Huntington's disease, dystonia, and epilepsy, among others.

In DBS, one or more probes are implanted in the basal ganglia area of the brain to administer electric pulses that curb Parkinson's symptoms (or the symptoms of the other diseases mentioned above). Although not fully understood, DBS is becoming a more and more widely accepted treatment, with various implantable devices currently being on the market. An example of such a device is the Active Therapy System sold by Medtronic, Inc. of Minneapolis, Minn. (www.medtronic.com/physician/activa/implantable.html). These devices, however, require the implantation of a relatively large battery and control pack in the chest with subcutaneous wires threaded up through the neck to the top of the skull and ultimately to the implanted probes (one or more). The control pack and wires are a common source of irritation and infection, sometimes necessitating long periods of antibiotics or even removal of the device. Furthermore, such devices are susceptible to a limited battery life and magnetic interference. After the average 3- to 5-year lifespan of an implant's battery, another surgery is required to replace the device. Thus, it would be advantageous to be able to provide DBS in a manner that eliminates the intrusive battery pack and wires, as well as the health risks commonly associated with them.

### SUMMARY OF THE INVENTION

The present invention relates to an apparatus for providing electrical stimulation to the brain of a patient for treating, for example, Parkinson's disease. The apparatus includes one or more probes for being implanted in the patient's brain and for providing electrical pulses to the brain. The apparatus also includes an implantable device for being implanted subcutaneously in the patient's head that has: (i) control circuitry adapted to generate the electrical pulses and provide the electrical pulses to the probes, and (ii) power circuitry for providing a DC power signal to the control circuitry. A power supply separate from the implantable device and external to the patient's body is also provided. The power supply provides power to the implantable device through a near-field technique, such as near-field inductive coupling, between the power supply and the power circuitry when the power circuitry is in proximity with the power supply. In particular, the power supply preferably includes an oscillator and a primary winding, wherein the oscillator generates a first AC signal and provides the first AC signal to the primary winding. The power circuitry includes a secondary winding, and the first AC signal induces a second AC signal in the secondary winding when the secondary winding is in proximity with the primary winding. The power circuitry converts the second AC signal into the DC power signal.

The control circuitry preferably includes a programmable processor and a wireless communications device. The programmable processor controls the generation of the electrical pulses based upon one or more pulse parameters. In this embodiment, the apparatus further includes a remote programming device external to the patient's body that is adapted to wirelessly transmit programming signals to the wireless communications device which are then provided to the programmable processor for adjusting the one or more pulse parameters. The one or more pulse parameters may specify one or more of a frequency of the electrical pulses, an amplitude of the electrical pulses, a pulse width of the electrical pulses, an on/off state of the electrical pulses, and an application location (i.e., to which electrodes) of the electrical pulses. The power supply may be provided as part of a piece of headgear, such as a hat or cap, to be worn by the patient.

A method of providing electrical stimulation to the brain of a patient is also provided that includes steps of implanting one or more probes into the brain, implanting a device subcutaneously in the patient's head, causing the device to generate electrical pulses and provide the electrical pulses to the one or more probes, and providing power to the device from a location external to the patient's body using a near-field technique, such as near-field inductive coupling. The method may further include selectively wirelessly adjusting the one or more pulse parameters from a second location external to the patient's body.

In an alternate embodiment, the present invention relates to an apparatus for providing electrical stimulation to the brain of a patient that includes one or more probes for being implanted in the brain and for providing electrical pulses to the brain, and an implantable device for being implanted subcutaneously in the patient's head. In this embodiment, the implantable device includes control circuitry electrically connected to the probes that is adapted to generate the electrical pulses and provide the electrical pulses to the probes, and power circuitry electrically connected to the control circuitry, wherein the power circuitry has an antenna for receiving energy transmitted in space from a far-field source. The power circuitry converts the received energy into a DC power signal and provides the DC power signal to the control circuitry. The energy transmitted in space may be RF energy transmitted by a remote RF source, such as a local radio station. Preferably, the antenna has an effective area greater than its physical area. The power circuitry may further include a matching network, such as an LC tank network having a non-zero resistance, electrically connected to the antenna and a voltage boosting and rectifying circuit, such as a charge pump, electrically connected to the matching network, wherein the received energy is an AC signal, and wherein the voltage boosting and rectifying circuit converts the AC signal into a DC signal. This embodiment does not include an energy storage device, such as a capacitor or rechargeable battery, for storing power for use when the antenna is not receiving the energy transmitted in space. This embodiment also preferably includes a remotely programmable processor.

In another alternative embodiment, the present invention relates to a method of providing electrical stimulation to the brain of a patient including the steps of implanting one or more probes into the brain, implanting a device subcutaneously in the patient's head, causing the device to generate electrical pulses and provide the electrical pulses to the probes, and providing power to the device by receiving energy transmitted in space from a remote far-field source external to the patient's body, such as a remote RF source like a local radio station, and converting the received energy into a DC power signal. The method may further include selectively wirelessly adjusting the one or more pulse parameters from a second location external to the patient's body.

In another embodiment, the invention relates to a method of treating a neurodegenerative disease, such as Parkinson's Disease, including steps of implanting a device in the head of a patient, causing the device to generate and provide electrical pulses to the brain, and providing power to the device from a location external to the patient's body. The power may be provided, for example, using a near field technique such as near-field inductive coupling, or by harvesting ambient energy from a far-field source, such as ambient RF energy from a far-field RF source.

It is an object of this invention to provide a method and apparatus for providing deep brain stimulation that does not require an onboard power supply that is implanted within the body of the patient.

It is a further object of this invention to provide a method and apparatus for providing deep brain stimulation that eliminates the problems associated with the subcutaneous wires that are associated with prior art devices.

It is still a further object of this invention to provide a method and apparatus for providing deep brain stimulation that eliminates the battery life and replacement problems associated with prior art devices.

It is still a further object of this invention to provide a method and apparatus for providing deep brain stimulation that is powered by a near-field technique, such as near-field inductive coupling.

It is still a further object of this invention to provide a method and apparatus for providing deep brain stimulation that is powered by harvesting ambient energy, such as ambient FR energy from a local radio station or other remote far-field source.

It is still a further object of this invention to provide a method and apparatus for providing deep brain stimulation that allows the electrical pulse parameters to be readily and non-intrusively adjusted from outside of the body.

It is still a further object of this invention to provide a method of treating a neurodegenerative disease such as Parkinson's Disease.

### BRIEF DESCRIPTION OF THE DRAWINGS

The accompanying drawings illustrate presently preferred embodiments of the invention, and together with the general description given above and the detailed description give below, serve to explain the principles of the invention. As shown throughout the drawings, like reference numerals designate like or corresponding parts.

FIG. 1 is a block diagram of a DBS device according to a first embodiment of the present invention;

FIG. 2 is a block diagram of control circuitry for driving the probes of the DBS device of FIG. 1 according to one embodiment of the invention;

FIG. 3 is a schematic illustration of the parameters used to specify the electrical pulses used in the present invention;

FIG. 4 is a block diagram of a remote programming device that allows an operator to set pulsing parameters for the DBS devices described herein; and

FIG. 5 is a block diagram of an implantable DBS device according to an alternative embodiment of the present invention.

### DESCRIPTION OF THE PREFERRED EMBODIMENTS

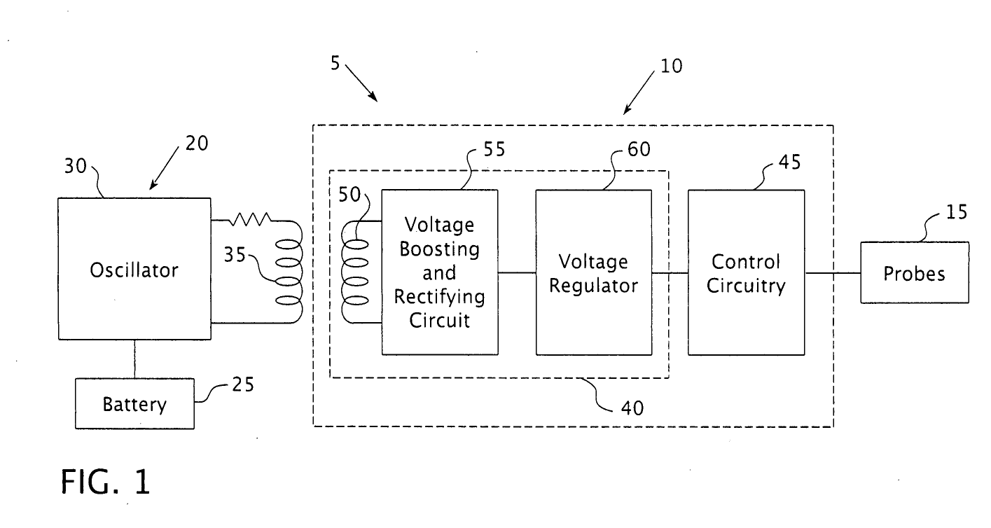

FIG. 1 is a block diagram of a DBS device 5 according to a first embodiment of the present invention for use in providing treatment to a patient, which preferably is a human, but may even include an animal. The DBS device 5 includes an implantable device 10 that is implanted subcutaneously in the head, i.e., it is mounted on the skull and below the skin. As such, the implantable device 10 may be implanted using only a local anesthetic. In addition, because the implantable device 10 is implanted in the body, the components thereof are provided on some type of biologically compatible substrate and encased in some type of biologically compatible material, such as a substrate or housing made from an accepted medical polymer. As described in greater detail herein, the implantable device 10 controls and drives one or more probes 15 which are implanted in the basal ganglia area of the brain by generating and providing to the probes 15 appropriate electrical pulses. The probes 15, in turn, administer the electrical pulses to the brain. Typically, each probe 15 is an elongated member that includes one or more electrodes along its length for actually applying the pulses to the brain. Because the electrodes are provided along the length of a probe 15, the electrical pulses can be provided at different depths within the brain.

As will be appreciated, the electronic components of the implantable device 10 require power in order to operate. The implantable device 10 does not, however, have an onboard power supply such as a battery. Instead, the embodiment of the implantable device 10 shown in FIG. 1 is remotely powered using a near-field technique, which in the embodiment shown in FIG. 1 is near-field inductive coupling. In the embodiment shown in FIG. 1 where near-field inductive coupling is used, the DBS device 5 includes a separate, external power supply 20 that is, in one particular embodiment, provided in headgear, such as a hat or cap, worn by the patient. The power supply 20 includes a battery 25 that is electrically connected to an adjustable oscillator 30 which generates an AC signal. A suitable example of an oscillator that may be used for the oscillator 30 is the LTC6900 precision low power oscillator sold by Linear Technology Corporation of Milpitas, Calif., which is capable of generating 50% duty cycle square waves at frequencies of between 1 KHz and 20 MHz. Other types/shapes of waveforms and/or duty cycles may also be used. The power supply also includes a primary winding 35 that is electrically connected to the oscillator 30 and receives the waveform generated thereby.

The implantable device 10 is provided with power circuitry 40 that provides a DC signal of an appropriate level for powering the control circuitry 45 provided as part of the implantable device 10. As described in greater detail herein, the control circuitry 45 controls the generation of the electrical pulses provided to the probes 15 (and ultimately to the patient's brain). As seen in FIG. 1, the power circuitry 40 includes a secondary winding 50, a voltage boosting and rectifying circuit 55 and a voltage regulator 60. In operation, when the AC signal is provided to the primary winding 35, a second AC signal is induced in the secondary winding 50 as a result of near-field inductive coupling with the primary winding 35.

Because of losses that occur in the inductive coupling, it is preferred to increase the voltage of the induced AC signal in order to provide a supply voltage of an appropriate level to the control circuitry 45 (as described hereinafter, the highest voltage necessary for the control circuitry 45 is typically 3 V, and the required voltage ranges from 1.5 V to 3 V, although voltages to 5 V may also be desired). In addition, because a DC signal is employed to power the control circuitry 45, the induced AC signal is also converted to DC. Thus, the induced AC signal is provided to the voltage boosting and rectifying circuit 55, which increases the voltage of and rectifies the received AC signal. In one particular embodiment, the voltage boosting and rectifying circuit 55 is a one or more stage charge pump, sometimes referred to as a “voltage multiplier.” Charge pumps are well known in the art. Basically, one stage of a charge pump essentially doubles the amplitude of an AC input voltage and stores the doubled DC voltage on an output capacitor. The voltage could also be stored using a rechargeable battery. Successive stages of a charge pump, if present, will essentially double the voltage from the previous stage. The DC signal that is output by the voltage boosting and rectifying circuit 55 is provided to a voltage regulator 60, which in turn provides a regulated DC voltage signal to the control circuitry 45. The voltage regulator 60 is primarily provided to resist spikes in the DC voltage signal provided to the control circuitry 45 and to resist DC voltage signals that may overdrive the control circuitry 45.

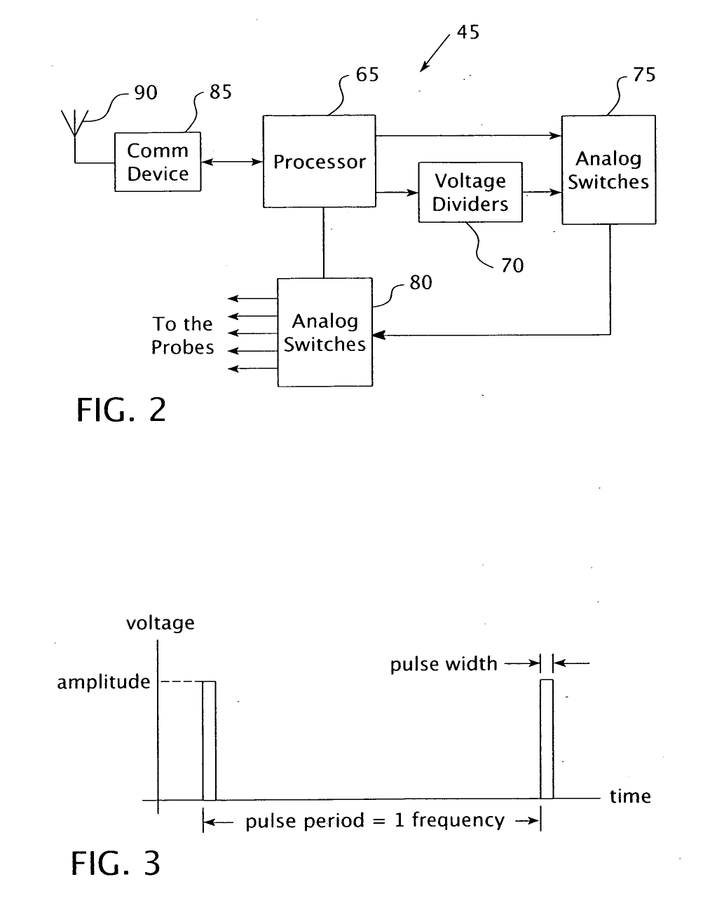

FIG. 2 is a block diagram of the control circuitry 45 for driving the probes 15 according to one embodiment of the invention. The control circuitry 45 includes a processor 65, such as a microcontroller or some other type of microprocessor. A suitable example of the processor 65 is the PIC16LF87 microcontroller sold by Microchip technology, Inc. of Chandler, Ariz. The processor 65 is programmed to output the actual pulses to be supplied to the probes 15, as well as determine to which electrode locations on the probes 15 the actual pulses are sent. As described elsewhere herein, a number of known DBS devices exist and therefore the required stimulation profile and range of parameters are well understood and are fairly standard according to medical practice. The nature of the pulses is determined by the following five parameters: (1) frequency, (2) amplitude, (3) pulse width, (4) on/off state (i.e., whether pulses are generated and/or provided to any electrodes at all), and (5) application location (i.e., to which particular electrodes the pulses are applied). These parameters are illustrated in FIG. 3. Current DBS devices have frequency, amplitude and pulse width ranges of about 2-185 Hz, 0-10.5 V, and 60-450 μs, respectively, although these complete ranges are not fully used. Typically, in DBS, the pulses administered to the brain are between 60 and 240 μs biphasic waveforms with a frequency of about 185 Hz. In addition, the pulses range in amplitude from about 1.5 V to 3 V, although normally the amplitude does not exceed 2.5 V.

In the particular embodiment of the DBS device 5 shown in FIGS. 1 and 2, the probes 15 include four electrodes for providing pulses to any one or any combination of four locations in the brain. In addition, in the particular embodiment of the DBS device 5 shown in FIGS. 1 and 2, the amplitude, frequency and pulse width of the pulses that are provided to the electrodes may be varied (four different amplitudes are possible). It will be appreciated, however, that this embodiment is meant to be exemplary only and that more or less probes and more or less voltage levels may be employed in a device without departing from the scope of the present invention. The actual pulses that are created and to which location or locations (i.e., which probes) they are provided is determined by parameters that, as noted above, are programmed in the processor 65. It is important in any DBS device for these parameters to be selectively adjustable, as the appropriate pulse frequency, amplitude and width must be selected and possibly later adjusted for each individual patient. Thus, the DBS device 5 of the present invention is, as described in greater detail herein, provided with a mechanism for selectively adjusting these parameters.

As stated above, the processor 65 (FIG. 2) creates and outputs pulses according to the selected pulse parameters. The circuitry in control circuitry 45 for adjusting the amplitude of the pulses as desired is realized by voltage dividers 70, which consist of four separate voltage dividers, one for each voltage level. Each voltage divider is driven by a direct pulse from the processor 65. The control circuitry 45 also includes a bank of analog switches 75 and a bank of analog switches 80. The processor 65 sends a signal to the bank of analog switches 75 to close a selected one of the switches to allow the output of a particular voltage divider (the chosen voltage level) to be passed through. In addition, the processor sends a signal to the bank of analog switches 80 to close those switches that are associated with the particular electrodes of probes 15 that are to receive the pulses.

According to an aspect of the present invention, the implantable device 10 is adapted to preserve power when pulsing is not required. Specifically, the processor 65 includes a watchdog timer, and the watchdog timer timeout, used as the wakeup mechanism, can be scaled down so that the processor 65 enters a sleep mode between pulses. In addition, a low power RC oscillator external to the processor 65 may be used with the processor 65 for clocking purposes such that its internal, high speed oscillator can be turned off to further persevere power.

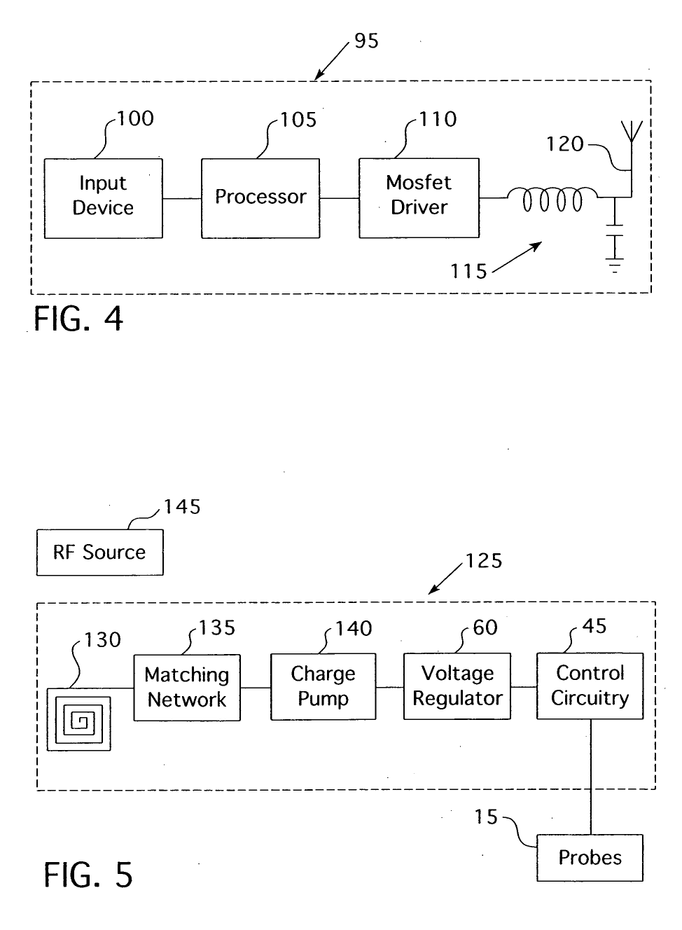

As noted above, it is preferred to be able to selectively adjust the pulsing parameters within the processor 65. Thus, according to a further aspect of the present invention, the DBS device 5 is provided with a mechanism for remotely and wirelessly programming the processor 65 so that the pulse parameters can be selectively adjusted. For this purpose, the control circuitry 45 includes a wireless communications device 85 having an antenna 90 that is in electronic communication with the processor 65 when it is necessary to perform adjustments. The wireless communications device 85 is adapted to receive programming signals sent from a remote programming device 95 shown in block diagram form in FIG. 4 and described hereinafter. The wireless communications device 85 may be any wireless receiver or transceiver that is able to communicate via any of a number of known wireless communications protocols, including, without limitation, an RF protocol such as Bluetooth. A suitable device that may be used for the wireless communications device 85 is the ATA5283 low power receiver that was sold by Atmel Corporation of San Jose, Calif. That particular device uses a simple ASK protocol at a frequency of 125 KHz and stays in a standby (low power sleep) mode until it senses a 125 KHz preamble of at least 5.64 ms, after which it wakes up and outputs digital data based on the presence of the 125 KHz signal. After data transmission, a simple digital high input to the reset pin puts the device back to sleep. The antenna used in this application is a small wire wrapped around the circuitry perimeter, although other forms are possible.

FIG. 4 is a block diagram of the remote programming device 95 that allows an operator to set pulsing parameters for the DBS device 5 and transmits programming signals which will cause the processor 65 to implement the selected parameters. The remote programming device 95 includes an input device 100 that enables an operator to set desired programming values. The input device 100 may be any suitable mechanism for inputting data, such as, without limitation, a keypad, a touch screen, or a series of slide switches. The input device 100 is in electronic communication with a processor 105 so that the data input by the operator can be sent thereto. The processor 105 is adapted to receive the input signals relating to the desired pulse parameters and convert them into programming signals appropriate for programming the processor 65 of the control circuitry 45. The processor 105 is preferably a microcontroller such as the PIC16LF87 microcontroller sold by Microchip technology, Inc. of Chandler, Ariz. Most suitable processors are not able to create a healthy sinusoid for transmitting the programming signals. As a result, in order to generate a signal appropriate for transmission, the processor 105 sends the programming signal pulses to a MOSFET driver 110, such as the TC4422 driver sold by Microchip corporation, provided as part of the remote programming device 95 which in turn drives an LC circuit 115 also provided as part of the remote programming device 95. The MOSFET driver 110 is powered by a separate 12 V power supply (not shown) so as to provide enough current to drive the high voltage and current oscillations in the LC circuit 115. In addition, the LC circuit 115 alone is not sufficient to send a strong signal to the control circuitry 45 (FIG. 1), but instead employs an antenna 120 to transmit the 125 KHz signal more efficiently. For this purpose, a PhidgetRFID antenna sold by Phidgets Inc, Calgary, Canada, designed for use with 125 KHz RFID systems, may be used for antenna 120. It will be appreciated that other suitable wireless transmitting devices, such as various commercially available transmitter and/or transceiver chips and antennas, may also be used without departing from the scope of the present invention.

FIG. 5 is a block diagram of an implantable DBS device 125 connected to implanted probes 15 according to an alternative embodiment of the present invention. The DBS device 125, like the DBS device 5, is adapted to be implanted subcutaneously in the head. In addition, the DBS device 125 does not have an onboard power supply such as a battery. Instead, the DBS device 125 is powered by harvesting energy that is transmitted in space. A number of methods and apparatus for harvesting energy from space and using the harvested energy to power an electronic device are described in U.S. Pat. No. 6,289,237, entitled “Apparatus for Energizing a Remote Station and Related Method,” U.S. Pat. No. 6,615,074, entitled “Apparatus for Energizing a Remote Station and Related Method,” U.S. Pat. No. 6,856,291, entitled “Energy Harvesting Circuits and Associated Methods,” and United States Patent Application Publication No. 2005/0030181, entitled “Antenna on a Wireless Untethered Device such as a Chip or Printed Circuit Board for Harvesting Energy from Space,” each assigned to the assignee hereof, the disclosures of which are incorporated herein by reference.

The DBS device 125 includes an antenna 130, which, in the embodiment shown in FIG. 5, is a square spiral antenna. The antenna 130 is electrically connected to a matching network 135, which in turn is electrically connected to a voltage boosting and rectifying circuit in the form of a charge pump 140. The charge pump 140 is electrically connected to a voltage regulator 60 which is electrically connected to the control circuitry 45. The control circuitry 45 is as described above in connection with FIG. 2 and controls the generation of the electrical pulses provided to the probes 15 (and ultimately to the patient's brain).

In operation, the antenna 130 receives energy, such as RF energy, that is transmitted in space by an RF source 145. The RF source 145 may be, without limitation, a local radio station. The RF energy received by the antenna 130 is provided, in the form of an AC signal, to the charge pump 140 through the matching network 135. The charge pump 140 amplifies and rectifies the received AC signal and provides the resulting DC signal to the voltage regulator 60. The voltage regulator 60 provides a regulated DC signal to the control circuitry 45 as a power supply. Thus, the DBS device 125 is able to be powered remotely without the need for an onboard power supply or energy storage device such as a capacitor or rechargeable battery.

The matching network 135 matches the impedance of the charge pump 140 to the impedance of the antenna 130 as complex conjugates for optimal antenna performance. In one particular embodiment, the matching network is an LC tank circuit formed by the inherent distributed inductance and inherent distributed capacitance of the conducing elements of the antenna 130. Such an LC tank circuit has a non-zero resistance R which results in the retransmission of some of the incident RF energy. This retransmission of energy may cause the effective area of the antenna 130 to be greater than the physical area of the antenna 130.

While preferred embodiments of the invention have been described and illustrated above, it should be understood that these are exemplary of the invention and are not to be considered as limiting. Additions, deletions, substitutions, and other modifications can be made without departing from the spirit or scope of the present invention. Accordingly, the invention is not to be considered as limited by the foregoing description but is only limited by the scope of the appended claims.

What is claimed is:

1. An apparatus for providing electrical stimulation to the brain of a patient, comprising:
one or more probes for being implanted in said brain and for providing electrical pulses to said brain;
an implantable device for being implanted subcutaneously in said patient's head, said implantable device having: (i) control circuitry electrically connected to said one or more probes, said control circuitry being adapted to generate said electrical pulses and provide said electrical pulses to said one or more probes, and (ii) power circuitry electrically connected to said control circuitry, said power circuitry providing a DC power signal to said control circuitry; and
a power supply separate from said implantable device and external to said patient's body, said power supply providing power to said implantable device through a near-field technique between said power supply and said power circuitry when said power circuitry is in proximity with said power supply.

2. The apparatus according to claim 1, wherein said near-field technique is near-field inductive coupling between said power supply and said power circuitry when said power circuitry is in proximity with said power supply.

3. The apparatus according to claim 2, wherein said power supply includes an oscillator and a primary winding, said oscillator generating a first AC signal and providing said first AC signal to said primary winding, wherein said power circuitry includes a secondary winding, wherein said first AC signal induces a second AC signal in said secondary winding when said secondary winding is in proximity with said primary winding, and wherein said power circuitry converts said second AC signal into said DC power signal.

4. The apparatus according to claim 3, wherein said power circuitry includes a voltage boosting and rectifying circuit that converts said second AC signal into a first DC signal, and a voltage regulator that receives said first DC signal and generates said DC power signal based thereon.

5. The apparatus according to claim 4, wherein said voltage boosting and rectifying circuit is a one or more stage charge pump.

6. The apparatus according to claim 1, wherein said control circuitry includes a programmable processor and a wireless communications device, said programmable processor controlling the generation of said electrical pulses based upon one or more pulse parameters, and wherein said apparatus further comprises a remote programming device external to said patient's body, said remote programming device being adapted to wirelessly transmit programming signals to said wireless communications device, said programming signals being provided to said programmable processor for adjusting said one or more pulse parameters.

7. The apparatus according to claim 6, wherein said one or more pulse parameters specify one or more of a frequency, an amplitude, a pulse width, an on/off state, and an application location of said electrical pulses.

8. The apparatus according to claim 1, wherein said power supply is provided as part of headgear to be worn by said patient.

9. The apparatus according to claim 8, wherein said headgear is one of a hat and a cap.

10. A method of providing electrical stimulation to the brain of a patient, comprising:
implanting one or more probes into said brain, said one or more probes being adapted to provide electrical pulses to said brain;
implanting a device subcutaneously in said patient's head, said device being electrically connected to said one or more probes;
causing said device to generate said electrical pulses and provide said electrical pulses to said one or more probes; and
providing power to said device from a location external to said patient's body using a near-field technique.

11. The method according to claim 10, wherein said near-field technique is near-field inductive coupling.

12. The method according to claim 11, wherein said step of providing power includes generating a first AC signal at said location external to said patient's body, said first AC signal inducing a second AC signal in said device, and converting said second AC signal to a DC power signal for powering said device.

13. The method according to claim 10, wherein said electrical pulses are generated based upon one or more pulse parameters, the method further comprising selectively wirelessly adjusting said one or more pulse parameters from a second location external to said patient's body.

14. The method according to claim 13, wherein said one or more pulse parameters specify one or more of a frequency, an amplitude, a pulse width, an on/off state, and an application location of said electrical pulses.

15. An apparatus for providing electrical stimulation to the brain of a patient, comprising:
one or more probes for being implanted in said brain and for providing electrical pulses to said brain; and
an implantable device for being implanted subcutaneously in said patient's head, said implantable device including:
control circuitry electrically connected to said one or more probes, said control circuitry being adapted to generate said electrical pulses and provide said electrical pulses to said one or more probes, and
power circuitry electrically connected to said control circuitry, said power circuitry having an antenna for receiving energy transmitted in space from a far-field source, said power circuitry converting said received energy into a DC power signal and providing said DC power signal to said control circuitry.

16. The apparatus according to claim 15, wherein said energy transmitted in space comprises RF energy transmitted by a remote RF source.

17. The apparatus according to claim 16, wherein said remote RF source is a radio station.

18. The apparatus according to claim 15, wherein said antenna has an effective area greater than its physical area.

19. The apparatus according to claim 18, wherein said power circuitry further includes a matching network electrically connected to said antenna and a voltage boosting and rectifying circuit electrically connected to said matching network, wherein said received energy is an AC signal, and wherein said voltage boosting and rectifying circuit converts said AC signal into a DC signal.

20. The apparatus according to claim 19, wherein said matching network is an LC tank network having a non-zero resistance.

21. The apparatus according to claim 19, wherein said voltage boosting and rectifying circuit is a one or more stage charge pump.

22. The apparatus according to claim 15, wherein said implantable device does not include an energy storage device for storing power for use when said antenna is not receiving said energy transmitted in space.

23. The apparatus according to claim 15, wherein said implantable device is contained entirely within said head and does not include any physical connections external to said head.

24. The apparatus according to claim 15, wherein said control circuitry includes a programmable processor and a wireless communications device, said programmable processor controlling the generation of said electrical pulses based upon one or more pulse parameters, and wherein said apparatus further comprises a remote programming device external to said patient's body, said remote programming device being adapted to wirelessly transmit programming signals to said wireless communications device, said programming signals being provided to said programmable processor for adjusting said one or more pulse parameters.

25. The apparatus according to claim 24, wherein said one or more pulse parameters specify one or more of a frequency, an amplitude, a pulse width, an on/off state, and an application location of said electrical pulses.

26. A method of providing electrical stimulation to the brain of a patient, comprising:
implanting one or more probes into said brain, said one or more probes being adapted to provide electrical pulses to said brain;
implanting a device subcutaneously in said patient's head, said device being electrically connected to said one or more probes;
causing said device to generate said electrical pulses and provide said electrical pulses to said one or more probes; and
providing power to said device by receiving energy transmitted in space from a remote far-field source external to said patient's body and converting said received energy into a DC power signal.

27. The method according to claim 26, wherein said energy transmitted in space comprises RF energy and wherein said remote source is a remote RF source.

28. The method according to claim 27, wherein said remote RF source is a radio station.

29. The method according to claim 26, wherein said electrical pulses are generated based upon one or more pulse parameters, the method further comprising selectively wirelessly adjusting said one or more pulse parameters from a second location external to said patient's body.

30. The method according to claim 29, wherein said one or more pulse parameters specify one or more of a frequency, an amplitude, a pulse width, an on/off state, and an application location of said electrical pulses.

31. A method of treating a neurodegenerative disease, comprising:
implanting a device in the head of a patient;
causing said device to generate and provide electrical pulses to said brain; and
providing power to said device from a location external to said patient's body.

32. The method according to claim 31, wherein said step of providing power to said device from a location external to said patient's body employs a near-field technique.

33. The method according to claim 32, wherein said near-field technique is near-field inductive coupling.

34. The method according to claim 32, wherein said step of providing power includes generating a first AC signal at said location external to said patient's body, said first AC signal inducing a second AC signal in said device, and converting said second AC signal to a DC power signal for powering said device.

35. The method according to claim 31, wherein said electrical pulses are generated based upon one or more pulse parameters, the method further comprising selectively wirelessly adjusting said one or more pulse parameters from a second location external to said patient's body.

36. The method according to claim 35, wherein said one or more pulse parameters specify one or more of a frequency, an amplitude, a pulse width, an on/off state, and an application location of said electrical pulses.

37. The method according to claim 31, wherein said step of providing power to said device from a location external to said patient's body includes receiving energy transmitted in space from a remote far-field source external to said patient's body and converting said received energy into a DC power signal.

38. The method according to claim 37, wherein said energy transmitted in space comprises RF energy and wherein said remote source is a remote RF source.

39. The method according to claim 38, wherein said remote RF source is a radio station.

40. The method according to claim 31, wherein said neurodegenerative disease is Parkinson's Disease.

41. The apparatus according to claim 1, wherein said implantable device is contained entirely within said head and does not include any physical connections external to said head.

42. The method according to claim 10, wherein said device is contained entirely within said head and does not include any physical connections external to said head.

43. The method according to claim 26, wherein said device is contained entirely within said head and does not include any physical connections external to said head.

44. The method according to claim 31, wherein said device is contained entirely within said head and does not include any physical connections external to said head.
